(19) United States
(12) Patent Application Publication
(10) Pub. No.: US 2023/0094611 A1
(43) Pub. Date: Mar. 30, 2023

(54) MEDICAL OR DENTAL HAND-HELD TREATMENT INSTRUMENT WITH A CAPACITIVE SENSOR

(71) Applicant: W&H Dentalwerk Bürmoos GmbH, Bürmoos (AT)

(72) Inventors: Christian Pruckner, Wien (AT); Tobias Schwarz, Wals (AT)

(73) Assignee: W&H Dentalwerk Bürmoos GmbH, Bürmoos (AT)

(21) Appl. No.: 17/207,999

(22) PCT Filed: Sep. 25, 2019
(86) PCT No.: PCT/EP2019/075778

§ 371 Date: Mar. 22, 2021

(30) Foreign Application Priority Data
Sep. 25, 2018 (EP) 18196500.5

(51) Int. Cl. A61C 1/07; A61B 17/22; A61B 90/92

(52) U.S. Cl. CPC A61C 1/07; A61B 17/22004; A61B 90/92; A61B 2017/00115

(57) ABSTRACT

A medical or dental, hand-held treatment instrument, comprising a single-part or multi-part outer sleeve, at least one controlling element having at least one capacitive sensor and a sensor surface provided on the outer sleeve and operatively connected to the capacitive sensor, wherein the controlling element generates an actuating signal when the sensor surface is touched or approached. The treatment instrument further comprises an indicating device, which is arranged on the treatment instrument and which indicates the sensor surface and/or the generation of an actuating signal due to a contact of the sensor surface or an approach to the sensor surface.

### CROSS REFERENCE TO RELATED APPLICATION

This application is a U.S. bypass continuation application of International Application No. PCT/EP2019/075778, filed Sep. 25, 2019, which in turn claims priority from European Patent Application No. 18196500.5, filed Sep. 25, 2018, now abandoned, which are incorporated herein by reference.

### FIELD

The present invention relates to a medical or dental treatment instrument which can be held by one hand and comprises a controlling element having a capacitive sensor provided on an outer sleeve.

### DESCRIPTION OF PRIOR ART

Such a medical or dental treatment instrument is known from WO 80/01643 A1.

A treatment instrument with a controlling element with a capacitive sensor provided on an outer sleeve has, among other things, the advantage that no operating button, switch or pushbutton is required on the surface of the outer sleeve. This eliminates, for example, the need to seal an opening in the outer sleeve in which such an operating button is disposed.

However, the absence of such an operating button poses the disadvantageous risk of operating the controlling element with a capacitive sensor unintentionally and/or unnoticed and thus triggering an undesired actuating process which may endanger the user or patient. This danger is particularly great if the controlling element with a capacitive sensor is provided on the treatment instrument, which can be held with one hand.

### SUMMARY

It is therefore an object to create a medical or dental treatment instrument which can be held by one hand and has a controlling element with a capacitive sensor provided on an outer sleeve, which does not have the disadvantages mentioned above. In particular, an unintentional and/or unnoticed operation of the controlling element with a capacitive sensor or an unintentional and/or unnoticed generation of an actuating signal is to be prevented.

According to an embodiment a medical or dental, hand-held treatment instrument comprises a single-part or multi-part outer sleeve and at least one controlling element provided on or in the outer sleeve, the controlling element having at least one capacitive sensor and a sensor surface provided on the outer sleeve and operatively connected to the capacitive sensor, wherein the controlling element generates an actuating signal when the sensor surface is touched or approached. The treatment instrument further comprises an indicating device disposed on (or in) the hand-held treatment instrument. The indicating device is configured to
(1) indicate the sensor surface operatively connected to the capacitive sensor, or
(2) indicate the generation of an actuation signal due to a contact with or approach to the sensor surface, or
(3) indicate the sensor surface operatively connected to the capacitive sensor and the generation of an actuation signal after touching or approaching the sensor surface.

By providing the indicating device, the risk of an unintentional and/or unnoticed operation of the controlling element with a capacitive sensor or an unintentional and/or unnoticed generation of an actuating signal is considerably reduced in an advantageous manner. As described in detail below, the indicating device is preferably configured to indicate the sensor surface and/or the generation of an actuating signal optically, acoustically and/or haptically or to generate and emit an optical, acoustical and/or haptical indication signal.

The medical or dental hand-held treatment instrument comprises in particular at least one of the following elements: a straight or curved handpiece; a contra-angle handpiece to which a tool can be attached at an angle to a longitudinal axis of a handle section; a handle element; a hand-held treatment instrument which is configured to deliver electromagnetic radiation to a treatment site; a hand-held treatment instrument which is configured to deliver a fluid to a treatment site; a hand-held treatment instrument which is intended to deliver a diagnostic and/or therapeutic medium to a treatment site; a hand-held treatment instrument which is intended to deliver vibrations, in particular ultrasonic vibrations, to a treatment site; a motor unit for driving a treatment tool acting on a treatment site, in particular with a pneumatically or electrically operated motor; a hand-held treatment instrument with an image recording device, in particular with a camera; a coupling element for connection to a media source, in particular a fluid source, a gas source or a source of electrical energy; an adapter unit.

The single-part or multi-part outer sleeve is made of metal or plastic, for example. The single-part or multi-part outer sleeve is preferably tubular and/or comprises a hollow interior, in particular for accommodating one or more components, for example parts of the indicating device. The single-part or multi-part outer sleeve preferably comprises (along its longitudinal axis) at least two sections which are arranged in an angled (with an angle >0°) or bent manner relative to one another.

The controlling element provided on the outer sleeve and having at least one capacitive sensor is preferably configured to switch an actuator on and off In particular, the actuator comprises at least one of the following elements: an electromagnetic radiation source, in particular an electromagnetic radiation source which emits light visible to humans and/or which emits electromagnetic radiation to detect an abnormality in tissue, for example caries and/or dental plaque; a motor drive, in particular an electric motor, an air motor or a vibration-generating motor, for example a piezomotor or a magnetostrictive motor; a conveying or conducting device, for example a pump or a valve, for a medium, in particular a medical, therapeutic or diagnostic treatment medium and/or a cooling medium, for example air, water, a powdery medium, a disinfectant, an anesthetic or a drug; an image recording device, in particular an image sensor, a CMOS or CCD sensor or a camera; a sensor for detecting a measured value, for example a temperature sensor, a pressure sensor, a conductivity sensor or a humidity sensor; a heating element, in particular for heating a medical, therapeutic or diagnostic treatment medium.

Alternatively or additionally, the controlling element provided on the outer sleeve and having at least one capacitive sensor is preferably configured to adjust an actuating value of an actuator continuously or in steps. The actuator preferably comprises at least one of the actuators mentioned in the foregoing. The actuating value comprises, for example, at least one of the following parameters: a power, for example of a motor drive, of a pump or of a heating element; an intensity or pulse rate of emitted radiation of an electromagnetic radiation source; a frequency, for example of generated and/or emitted oscillations; a volumetric flow, for example of a medical, therapeutic or diagnostic treatment medium and/or of a cooling medium; a focal length or a magnification factor of an optical device, in particular of a camera; a temperature, for example of a heating element.

The at least one capacitive sensor is configured in a known manner to operate, in particular to generate an actuation signal, on the basis of the change in electrical capacitance of one or more capacitors and/or of the treatment instrument. Preferably, the at least one capacitive sensor comprises rigid and/or immovable plates whose capacitance changes due to an approach or a contact by an operating element. The operating element comprises, for example, an electrically conductive material or dielectric, in particular a hand or finger of a user.

The sensor surface, whose capacitance changes when it is touched or approached so that the at least one capacitive sensor generates an actuation signal, is configured as part of the at least one capacitive sensor and/or is operatively connected to it. The sensor surface is in particular integrally formed with the outer sleeve or is formed in one part with the outer sleeve. In particular, the sensor surface is formed by a part or section of the single-part or multi-part outer sleeve. This advantageously avoids accumulation of dirt at the edges on the surface of the outer sleeve and sealing of the sensor surface.

Alternatively, it is also possible to configure the sensor surface as a separate component which is integrated, in particular flush, into the single-part or multi-part outer sleeve. The sensor surface then comprises, for example, a glass surface, a plastic surface, a ceramic surface and/or a metal surface or metal foil. Preferably, an opening is provided in the outer sleeve in which the sensor surface is accommodated. Preferably, a sealing element is provided to seal the opening in which the sensor surface is accommodated.

Particularly preferably, only a part or section of the outer sleeve is configured as a controlling element and/or sensor surface. In particular, the controlling element and/or the sensor surface has/have a smaller area than the total area of the outer sleeve of the treatment instrument. This advantageously further reduces the risk of unintentional and/or unnoticed operation of the controlling element.

Preferably, an electrical supply line is provided to connect the at least one capacitive sensor to an electrical energy source and/or to supply it with electrical energy.

Preferably, a signal line, in particular an electrical signal line, is provided which connects the at least one capacitive sensor and an electronic control system which is configured to receive and evaluate the actuating signal of the capacitive sensor. In particular, the signal line is configured to transmit the actuation signal of the at least one capacitive sensor to the control electronics.

Preferably, the electrical supply line and/or signal line is additionally configured to transmit electrical energy to an electrical load provided in or connected to the treatment instrument, for example an electromagnetic radiation source. This advantageously achieves a reduction of the electrical lines of the treatment instrument.

Preferably, the at least one capacitive sensor, the sensor surface, the electrical supply line, the signal line and the control electronics form an electrical circuit for setting or switching an actuator.

Preferably, at least a part of the indicating device, in particular a marking or an indication surface of the indicating device that emits an indication signal, covers or forms a part of the sensor surface. Alternatively, at least a part of the indicating device, in particular a marking or an indication surface of the indicating device that emits an indication signal, covers or forms the entire sensor surface. Preferably, at least a part of the indicating device, in particular a marking or an indication surface of the indicating device emitting an indication signal, is arranged on a part of the sensor surface or the entire sensor surface. Thus, in an advantageous manner, the position of the sensor surface and/or its actuation is indicated directly on the sensor surface itself and/or by the sensor surface.

Particularly preferably, the sensor surface and a marking or an indication surface of the indicating device that emits an indication signal, in particular a light emitting surface, are of the same size and/or identical, i.e., formed by the same component. Particularly preferably, a transparent plastic or glass surface or a metallic surface forms the identical sensor surface and marking or indication surface. Preferably, the indication surface comprises a light guide forming at least part of the sensor surface. This advantageously simplifies the manufacture of the treatment instrument, since a common component is sufficient for the sensor surface and marking or indication surface.

Alternatively, the sensor surface and an indication surface of the indicating device emitting an indication signal, in particular a light emitting surface, are separated and/or spaced apart and/or form two separate surfaces on the surface of the outer sleeve of the treatment instrument. In particular, at least a portion of the indicating device surrounds the sensor surface, preferably directly adjacent thereto. For example, the indication surface, in particular of a light emitting indicating device, is arranged annularly around the sensor surface. This advantageously improves the perceptibility of the indicating device, in particular of a light-emitting indicating device.

Preferably, an indication surface of the indicating device that emits an indication signal, in particular a light emitting surface or a vibration emitting surface, is larger than the sensor surface. Preferably, the sensor surface is formed as part of this larger indication surface. This advantageously improves the perceptibility of the indicating device.

Preferably, the indicating device covers or comprises only a portion of the outer sleeve of the hand-held treatment instrument.

Preferably, at least a part of the indicating device is operatively connected to at least one section of the outer sleeve, preferably to at least one section of the sensor surface, in particular connected in a signal-transmitting manner, particularly preferably in a vibration-transmitting or light-conducting manner, for example via a vibrating rod or a light guide. In this way, it is advantageously possible to transmit the indication signal generated by the indicating device to the sensor surface in a targeted manner.

Preferably, the controlling element and/or the at least one capacitive sensor and/or the indicating device is/are formed on only one section of the outer sleeve of the treatment instrument, in particular on a coupling or connecting section for connection to a media, energy source and/or control or regulating device or on an accessory holding section to which an accessory, for example a tool, a light guide or a nozzle, can be attached. Advantageously, this further reduces the risk of unintentional and/or unnoticed operation of the controlling element.

Preferably, the indicating device comprises a marking on the surface of the outer sleeve and/or the controlling element and/or the sensor surface that can be seen and/or felt by the user. Preferably, the marking is planar or surrounds the sensor surface in the form of a frame. This allows the user to directly recognize the position of the controlling element and/or the sensor surface in an advantageous manner.

Preferably, the marking comprises a color marking. Particularly preferably, the color marking is provided on the single-part or multi-part outer sleeve of the treatment instrument, in particular on its surface (visible to the user) or outer side. In particular, the color of the color marking is different from the color of the outer sleeve. This makes the marking particularly easily recognizable for the user in an advantageous manner.

Preferably, the color marking comprises a label that can be attached, in particular adhered or glued, to the outer sleeve. Alternatively, the color marking comprises a paint or varnish that can be applied to the outer sleeve. Alternatively, the color marking comprises a color change of the single-part or multi-part outer sleeve of the treatment instrument, in particular its surface or outer side, caused by a chemical process, for example by oxidation, anodizing, galvanizing or nitriding. Alternatively, the color marking comprises a color change of the single-part or multi-part, in particular metallic, outer sleeve of the treatment instrument, in particular its surface or outer side, by laser treatment. Alternatively, the color marking comprises a colored window, for example made of glass or plastic, which is accommodated in an opening of the outer sleeve of the treatment instrument and is preferably also configured as a sensor surface. Alternatively, the color marking comprises color pigments which are inserted and/or accommodated in a material of the outer sleeve, for example plastic.

Preferably, the marking comprises a haptically perceptible marking for a user, which for haptic perception comprises at least one depression, groove, slot, extension, projection and/or elevation. The haptically perceptible marking is formed, for example, by mechanical processing and/or by erosion in the surface of the outer sleeve and/or the controlling element and/or the sensor surface. The haptically perceptible marking comprises, for example, corrugation. A haptically perceptible marking is advantageously perceptible to the user without the user having to look at the marking.

Preferably, the marking is attached to or applied to the outer sleeve or formed as part of the outer sleeve or by the outer sleeve or on or in the outer sleeve.

Preferably, the indicating device is configured to generate vibrations, in particular to indicate the generation of an actuating signal due to a contact with the sensor surface or approach to the sensor surface. In an advantageous manner, it is thus not necessary for the user to look at the indicating device in order to perceive a signal emitted by the indicating device.

In particular, the indicating device for generating vibrations comprises a vibration generator, for example at least one piezo element, in particular a piezo stack, and/or an, in particular electric, unbalance motor. For generating the vibrations, the unbalanced motor comprises in particular a mass arranged eccentrically to an axis of rotation of a rotor shaft.

Preferably, the indicating device for generating vibrations is configured to transmit the vibrations generated by the vibration generator in the form of structure-borne sound to an indication surface of the indicating device, which emits an indication signal, and/or to the sensor surface. The transmission of the vibrations to the indication surface or sensor surface is carried out in particular directly by the vibration generator itself and/or via at least one further component of the indicating device, for example a vibrating rod, and/or via at least one component of the treatment instrument, for example the outer sleeve, a circuit board, a media line, a bearing or a shaft. In this way, the indication signal can be perceived in an advantageous manner by the user, predominantly haptically, and is thus easily distinguishable, particularly in an environment with many acoustic signals.

Alternatively or additionally, the indicating device is configured to generate vibrations that can be perceived acoustically by a user. The indicating device preferably comprises at least one component that can be set in vibration, for example the vibration generator itself or a vibrating rod connected thereto, which emits vibrations to the air and/or to a component of the treatment instrument, for example the outer sleeve, the sensor surface, the controlling element, a media line, a bearing or a shaft, in order to generate airborne sound.

Preferably, the indicating device is configured to produce a deformation of the sensor surface that can be perceived by a user, in particular to indicate the generation of an actuating signal due to a contact with the sensor surface or an approach to the sensor surface. The deformation comprises in particular a bending or arching of the sensor surface. This means that the indication signal can be perceived by the user in a predominantly haptic manner and is therefore easily distinguishable, particularly in an environment with many acoustic signals.

The sensor surface is configured in particular as a deformable or bendable sensor surface, for example as a plastic surface, which is made in particular from polymethyl methacrylate.

Preferably, the indicating device comprises at least one piezoelectric element arranged on or operatively connected to the sensor surface. The at least one piezoelectric element is configured to perform a mechanical movement when an electrical voltage is applied, which movement is transmitted to the sensor surface and deforms or bends it. After termination of the application of the electrical voltage, the at least one piezoelectric element and the sensor surface return to their initial position.

Preferably, the indicating device is configured to emit light that is visible to a user, in particular in order to indicate the generation of an actuation signal due to a contact with the sensor surface or an approach to the sensor surface. This advantageously creates an indicating device whose indication signal can he perceived independently of touching the treatment instrument, in particular the sensor surface.

Preferably, the indicating device, which emits a light visible to a user, comprises at least one of the following elements: a light source, in particular a light bulb or one or more optical semiconductor elements, for example an LED; an optical light guide, for example a glass rod or a glass fiber rod; an optically transparent light emitting surface. Preferably, the light source and the optical light guide and/or the optically transparent light-emitting surface are connected to each other in a light-conducting manner, so that the visible light emitted by the light source can be guided to and into the optically transparent light-emitting surface.

Preferably, the optically transparent light emitting surface forms at least part of the sensor surface or the entire sensor surface. Alternatively or additionally, the optically transparent light emitting surface surrounds the sensor surface, in particular annularly. Preferably, the optically transparent light emitting surface is made of glass or plastic, for example polymethyl methacrylate. This means that the user only has to concentrate his perception on a single point in order to actuate the controlling element and to detect the indication signal of the indicating device in an advantageous manner.

Preferably, the visible, and in particular colored, light emitted by the indicating device passes through the (optically transparent) sensor surface or through the light emitting surface separated or spaced from the sensor surface to indicate to the user the generation of an actuating signal.

Preferably, a circuit board is disposed inside the treatment instrument on which at least a portion of the indicating device is disposed. Alternatively, the outer sleeve is made of plastic and its inner side with a metallic coating arranged thereon, in particular a copper coating, forms a circuit board.

Preferably, for example, the vibration generator described in the foregoing or the at least one light source described in the foregoing is arranged on the circuit board. Preferably, the circuit board is configured as a flexible, bendable circuit board. Preferably, at least a part of the controlling element, in particular at least a part of the capacitive sensor, is also arranged on the circuit board. This advantageously creates a compact structure of the treatment instrument.

Preferably, the medical or dental treatment instrument that can be held with one hand comprises control electronics, in particular arranged on the circuit board, which are connected to the capacitive sensor and the indicating device and are configured to receive the actuation signal of the capacitive sensor, to process it if necessary, and to control the indicating device on the basis of the received actuation signal in order to indicate optically, acoustically and/or haptically a contact with the sensor surface or an approach to the sensor surface. Thus, in an advantageous manner, a treatment instrument is created which compactly comprises an electrical circuit comprising the capacitive sensor, the sensor surface, the indicating device and the control electronics. In particular, the control electronics is configured to generate a control signal based on the received actuation signal to control the indicating device. In particular, the indicating device is configured to receive the control signal and to generate and output an optical, acoustic and/or haptic indication signal based on the received control signal.

Preferably, the control electronics of the medical or dental hand-held treatment instrument is connected to an actuator and is configured to control the actuator, in particular one of the actuators mentioned in the foregoing, on the basis of the received actuating signal of the capacitive sensor. Preferably, the control electronics comprise a controller, for example to facilitate data transmission. Preferably, the actuator is part of and/or arranged on the treatment instrument. Preferably, controlling by the control electronics comprises switching the actuator on and off and/or setting an actuating value of an actuator steplessly or in steps, as described in the foregoing. Advantageously, this provides a treatment instrument with an independent circuit for the indicating device and the actuator.

Preferably, the medical or dental hand-held treatment instrument comprises a transmission device which is configured to transmit the actuation signal or a control signal triggered by the actuation signal to an external (i.e., outside the hand-held treatment instrument) controller. Thus, it is advantageously possible to process, store and/or display the actuating signal and/or the control signal outside the hand-held treatment instrument. Preferably, the external controller is provided in a dental unit or in a tabletop control unit.

In particular, the external controller is configured to receive the actuating signal of the capacitive sensor, to process it if necessary, and to control the indicating device of the treatment instrument on the basis of the received actuating signal in order to indicate optically, acoustically and/or haptically a contact with the sensor surface or an approach to the sensor surface. Alternatively or additionally, the external controller is connected to an actuator and is configured to control the actuator, in particular one of the actuators mentioned in the foregoing and provided on the treatment instrument, based on the received actuating signal of the capacitive sensor or a control signal triggered thereby. Alternatively or additionally, the external controller is configured to control, on the basis of the received actuating signal of the capacitive sensor or a control signal triggered thereby, an external display device provided on the external controller or the dental unit or the table control unit, for example in order to display the setting of the actuator.

Preferably, the transmission device comprises at least one of the following: a coupling device, in particular a mechanical coupling device; a hose; an electrical or optical line; a cable; a wireless transmitter; a radio communication.

Preferably, the optical, acoustic and/or haptic indication signals described above are limited in time by the control electronics or the controller, in particular to a period of less than one second or a few seconds, for example in a range of 0.1 sec-5 sec.

These and other embodiments will be described below with reference to the following drawings.

### BRIEF DESCRIPTION OF THE DRAWINGS

FIG. 1 shows a first embodiment of a medical or dental hand-held treatment instrument having a controlling element with a capacitive sensor and an indicating device indicating the sensor surface of the capacitive sensor and the generation of an actuation signal;

FIG. 2 shows a second embodiment of a medical or dental hand-held treatment instrument having a controlling element with a capacitive sensor and an indicating device in the form of markings indicating the sensor surface of the capacitive sensor;

FIG. 3 shows a third embodiment of a medical or dental hand-held treatment instrument having a controlling element with a capacitive sensor and an indicating device with a vibration generator, which indicates the sensor surface of the capacitive sensor and the generation of an actuating signal;

FIGS. 4A and 4B show a fourth embodiment of a medical or dental hand-held treatment instrument having a controlling element with a capacitive sensor and an optical indicating device indicating the sensor surface of the capacitive sensor and the generation of an actuating signal;

FIG. 5 shows a fifth embodiment of a medical or dental hand-held treatment instrument having a controlling element with a capacitive sensor and an indicating device indicating the sensor surface of the capacitive sensor and the generation of an actuation signal by deformation of the sensor surface;

FIG. 6 shows in a block diagram an embodiment of a signal transmission and signal processing of a medical or dental hand-held treatment instrument having a controlling element with a capacitive sensor and an indicating device indicating the generation of an actuating signal;

FIG. 7 shows in a block diagram an alternative embodiment of a signal transmission and signal processing of a medical or dental hand-held treatment instrument having a controlling element with a capacitive sensor and an indicating device indicating the generation of an actuating signal.

### DETAILED DESCRIPTION

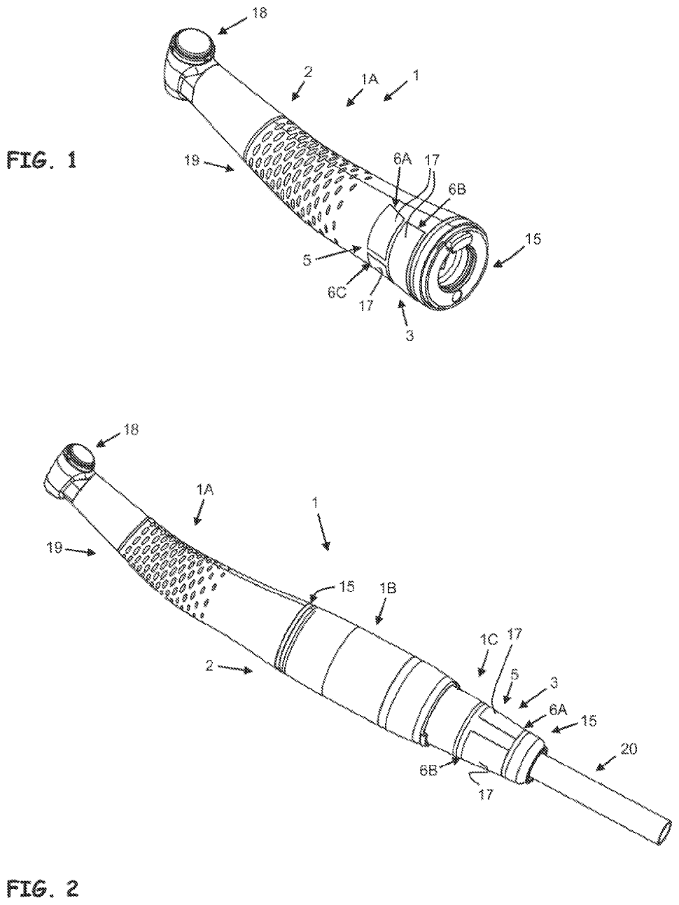

The medical or dental treatment instrument 1 shown in FIG. 1, which can be held by one hand, is configured as a contra-angle handpiece 1A. It comprises a head part 18 and an adjoining curved handle part 19. At least a tool holder is movably arranged in the head part 18 in order to detachably receive a medical or dental treatment tool. Furthermore, a drive device is provided in the head part 18 and/or handle part 19, which comprises, for example, one or more shafts, a gearbox, a compressed gas line, an impeller that can be driven by compressed gas, or the like, The drive device is operatively connected to the tool holder to set the tool holder and the tool held therein in motion.

The treatment instrument 1 comprises a multi-piece outer sleeve 2, which comprises a head sleeve portion provided at the head portion 18 and a curved handle sleeve portion provided at the handle portion 19.

A coupling device or transmission device 15 is provided at the end of the treatment instrument 1 facing away from the head part 18, which can be detachably connected to a supply unit or dental unit or an external controller 16 (see FIGS. 6, 7). At least one medium, for example electrical energy, compressed gas, in particular compressed air, water, light and/or data, for example measurement data, control or regulating data, identification data, for example for identifying the treatment instrument 1 or the tool connected thereto, operating data or similar data, can be transmitted via the coupling device 15. The transmission of the at least one medium and/or the data between the treatment instrument 1 and the supply unit or dental unit or the external controller 16 can be unidirectional or bidirectional.

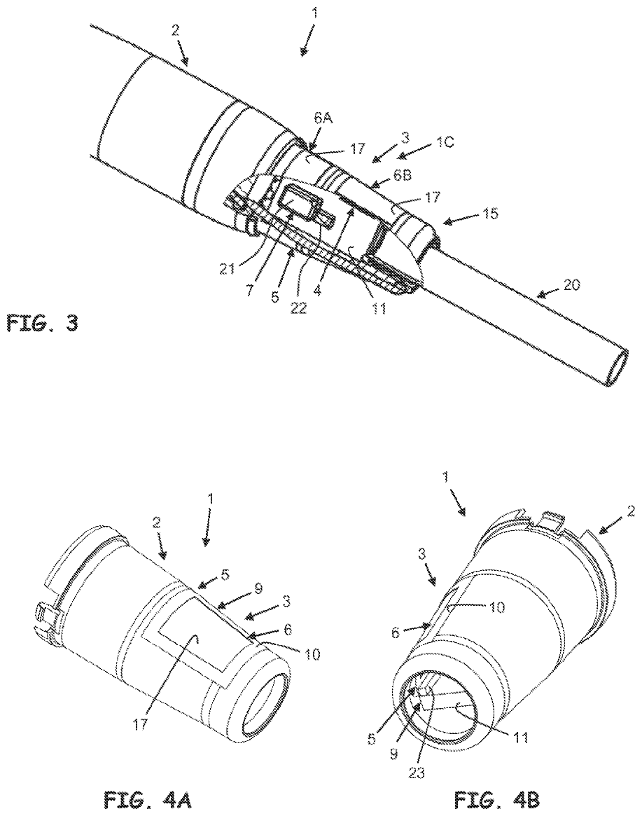

A controlling element 3 is provided on the outer sleeve 2 on the handle part 19, in particular on the end of the handle part 19 having the coupling device 15, or on the coupling device 15. The controlling element 3 comprises a plurality of capacitive sensors 4 (see FIG. 3) and three sensor surfaces 6A, 6B, 6C which are provided on the outer sleeve 2 and are operatively connected to the capacitive sensors 4. When the sensor surfaces 6A, 6B, 6C are touched or approached, the controlling element 3 generates actuating signals in order to control one or more actuators 13 (see FIGS. 6, 7).

Exemplarily, the sensor surface 6A is provided for switching on and off a first actuator 13, for example a motor for driving a tool connectable to the treatment instrument 1. Exemplarily, the sensor surface 6B is provided for setting a parameter of the first actuator 13, for example the speed or power of the motor, continuously or in predetermined steps. Exemplarily, the sensor surface 6C is provided for switching on and off a second actuator, for example an illumination device of the treatment instrument 1 for emitting electromagnetic radiation in the direction of the treatment site.

The sensor surfaces 6A, 6B, 6C are formed as a part or section of the outer sleeve 2, in particular in one piece with the outer sleeve 2. Thus, the sensor surfaces 6A, 6B, 6C and/or the boundaries of the sensor surfaces 6A, 6B, 6C are difficult or impossible for a user to recognize when looking at the outer sleeve 2, and reliable operability of the controlling element 3 is not ensured.

Therefore, in order to make the sensor surfaces 6A, 6B, 6C recognizable to a user, an indicating device 5 is provided on the treatment instrument 1, which indicates the sensor surface 6 and/or its outer boundaries. The indicating device 5 comprises three markings 17, for example color markings, which are arranged around, on or above the sensor surfaces 6A, 6B, 6C on the outer sleeve 2, wherein one marking 17 each is assigned to one of the three sensor surfaces 6A, 6B, 6C. This enables reliable operability of the controlling element 3 and the three sensor surfaces 6A, 6B, 6C.

The indicating device 5 of the treatment instrument 1 is further provided for indicating the generation of an actuating signal due to a contact of or an approach to the sensor surfaces 6A, 6B, 6C. Thus, the user receives feedback as to whether or not the controlling element 3 has generated an actuating signal. For this purpose, the indicating device 5 generates an optical, acoustic and/or haptic indicating signal and emits it, in particular to the sensor surfaces 6A, 6B, 6C, particularly preferably only to the sensor surface 6A, 6B, 6C used by the user during the positioning process. The indicating device 5 can in particular be configured as described in more detail below with reference to FIGS. 3-5 and have the components mentioned therein.

The medical or dental hand-held treatment instrument 1 shown in FIG. 2 comprises a contra-angel handpiece 1A, a motor drive 1B and a coupling unit 1C.

The contra-angle handpiece 1A comprises a head portion 18, a handle portion 19, and a coupling device or transmission device 15, and is similar in these components to the contra-angle 1A of FIG. 1, to the description of which reference is made. However, the contra-angle handpiece 1A of FIG. 2 does not have a controlling element 3 and an indicating device 5.

The motor drive 1B is configured as a separate component that can be detachably connected to the coupling device 15 of the contra-angle handpiece 1A via one or more coupling elements (on the side facing the contra-angle handpiece). Alternatively, it is also possible to form the motor drive 1B as an integral part of the contra-angle handpiece 1A that cannot be removed by the user. The motor drive 1B comprises, in particular, an electric motor which can be connected to shafts and/or gears arranged in the contra-angle handpiece 1A in order to set the latter and a treatment tool connectable thereto in a drive movement.

At its end facing away from the contra-angle handpiece 1A, the motor drive 1B is connected to the coupling unit or hose coupling 1C. The coupling unit 1C is configured as a transmission device 15 which connects the contra-angle handpiece 1A and the motor drive 1B via a hose 20 to a control and/or regulating and/or supply unit or dental unit or an external controller 16 for the purpose of exchanging data, energy and/or media. One or more electrical and/or optical lines and/or media lines are arranged in the hose 20 for transmitting the data, energy and/or media.

Furthermore, a controlling element 3 is arranged on the outer sleeve 2 having at least one capacitive sensor 4 and two sensor surfaces 6A, 6B provided on the outer sleeve 2 of the coupling unit 1C and operatively connected to the at least one capacitive sensor 4. When the sensor surfaces 6A, 6B are touched or approached by an operating element, for example a finger of a user, the controlling element 3 generates actuating signals in order to control one or more actuators 13.

An indicating device 5 comprises two markings 17, for example color markings or haptically perceptible markings, which are arranged around, on or above the sensor surfaces 6A, 6B or are formed as part of the sensor surfaces 6A, 6B. One marking 17 is assigned to each of the two sensor surfaces 6A, 6B. This enables reliable operability of the controlling element 3 and the sensor surfaces 6A, 6B.

The medical or dental, hand-held treatment instrument 1 shown in FIG. 3 in turn comprises a coupling unit 1C, which is configured in particular as a transmission device 15 for transmitting data, energy and/or media from and/or to a control and/or regulating and/or supply unit or dental unit or an external controller 16.

The coupling unit 1C comprises a controlling element 3 having at least one capacitive sensor 4 and a plurality of sensor surfaces 6A, 6B provided on the outer sleeve 2 and operatively connected to the at least one capacitive sensor 4. An indicating device 5 comprises markings 17, for example color markings or haptically perceptible markings, which are arranged around, on or above the sensor surfaces 6A, 6B or are formed as part of the sensor surfaces 6A, 6B in order to indicate the sensor surfaces 6A, 6B and/or their outer boundaries. One marking 17 is assigned to each of the sensor surfaces 6A, 6B.

In addition, the indicating device 5 is provided to indicate the generation of an actuating signal due to a touch of the sensor surfaces 6A, 6B or an approach to them. For this purpose, the indicating device 5 generates a haptic indicating signal and outputs it, in particular to the sensor surfaces 6A, 6B, particularly preferably only to the sensor surface 6A, 6B used by the user during the positioning process.

The haptic indicating device 5 comprises a vibration exciter 7. The vibration exciter 7 comprises an unbalance motor with an electric motor 21 and a mass 22 arranged eccentrically to an axis of rotation of a rotor shaft of the electric motor 21.

The vibration exciter 7 is mounted on a circuit board 11 inside the coupling unit 1C. The circuit board 11 is directly or indirectly connected to the inside of the outer sleeve 2 of the coupling unit 1C, so that the vibrations generated by the vibration exciter 7 can be transmitted via the circuit board 11 to the outer sleeve 2, in particular to the sensor surfaces 6A, 6B, and can be perceived by the user. The user thus receives haptic feedback on the actuation of the controlling element 3 and/or the generation of an actuating signal.

FIGS. 4A, 4B show an outer sleeve 2 of a medical or dental hand-held treatment instrument 1. A controlling element 3 having at least one capacitive sensor 4 and a sensor surface 6 provided on the outer sleeve 2 and operatively connected to the at least one capacitive sensor 4 is provided on the outer sleeve 2.

An indicating device 5 comprises a marking 17 arranged on or formed as part of the sensor surface 6 to indicate the sensor surfaces 6 and/or their outer boundaries. The indicating device 5 is also provided for indicating the generation of an actuating signal due to a contact with or an approach to the sensor surface 6. For this purpose, the indicating device 5 is configured as an optical display device 9 and generates an optical display signal, in particular light visible to the user. The visible light emerges from the outer sleeve 2 to the outside through a light emitting surface 10 which is separate from and adjacent to the sensor surface 6.

The light emitting surface 10 is formed of glass or plastic and surrounds the sensor surface 6 and/or the capacitive sensor 4 in the form of a frame. The light emitting surface 10 and the sensor surface 6 are received in an opening of the outer sleeve 2, in particular flush.

The optical display device 9 comprises a light source 23, in particular at least one optical semiconductor element, for example an which is mounted on a circuit board 11 and can be supplied with electrical energy via the circuit board 11. At least a portion of the visible light is emitted from the light source 23 in the direction of the light emitting surface 10 to be guided outwardly through the light emitting surface 10.

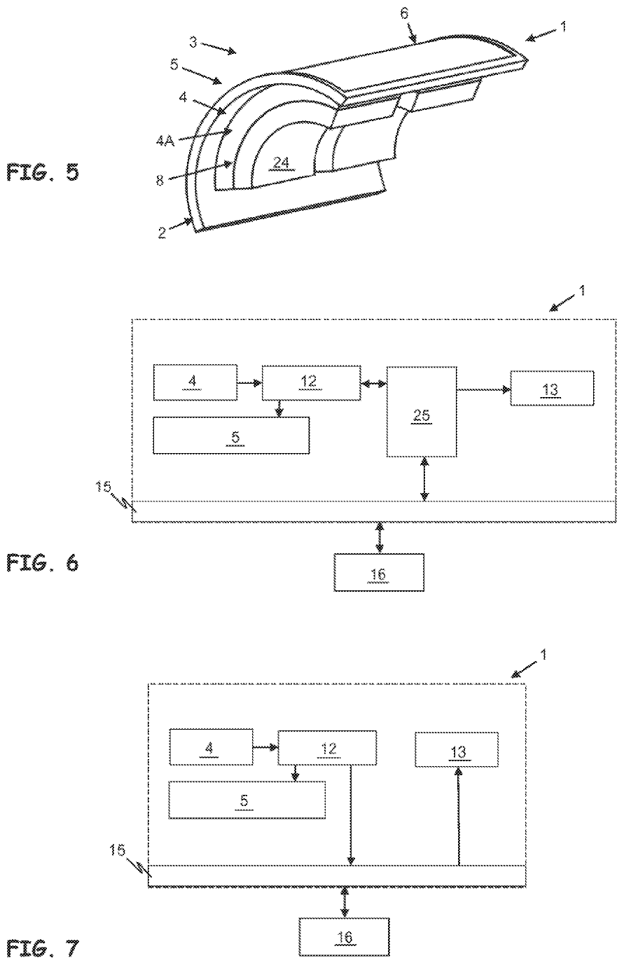

FIG. 5 shows an outer sleeve 2 of a medical or dental hand-held treatment instrument 1. A controlling element 3 is provided on the outer sleeve 2 having at least one capacitive sensor 4 and a sensor surface 6 provided on the outer sleeve 2 and operatively connected to the at least one capacitive sensor 4.

The capacitive sensor 4 comprises a metal layer 4A, for example a copper layer, as may also apply, for example, to the capacitive sensors 4 of FIGS. 1-4B. The metal layer 4A is arranged on the inside of the sensor surface 6, for example through vapor-deposition.

An indicating device 5, 8 is configured to indicate the generation of an actuating signal due to a contact with the sensor surface 6 or an approach thereto by a deformation of the sensor surface 6 perceptible to a user. For this purpose, at least one piezoelectric element 24 is provided, which is arranged on the metal layer 4A and thus operatively connected to the sensor surface 6. The at least one piezoelectric element 24 is configured to perform a mechanical movement when an electrical voltage is applied, which movement is transmitted to the sensor surface 6 and deforms or bends it.

FIGS. 6 and 7 schematically show two different embodiments of a signal transmission and signal processing of medical or dental hand-held treatment instruments 1, each having a controlling element 3 with a capacitive sensor 4 and an indicating device 5 indicating the generation of an actuating signal. The indicating device 5 is preferably formed by one of the indicating devices 5 shown in FIGS. 1-5 and described in the associated description.

Numeral 12 represents control electronics arranged in the treatment instrument 1, which are connected to the capacitive sensor 4 and the indicating device 5 and are configured to receive the actuating signal of the capacitive sensor 4 and to control the indicating device 5 on the basis of the received actuating signal in order to indicate contact with the sensor surface 6-6C or an approach thereto. The activation of the indicating device 5, for example the emission of visible light or of vibrations or the execution of a movement to cause in particular a deformation of the sensor surface 6, is thus achieved in both embodiments of FIGS. 6 and 7 by the control electronics 12 arranged in the treatment instrument 1, in particular on the circuit board 11.

According to FIG. 7, the treatment instrument 1, in particular the coupling device or transmission device 15, is configured to transmit the actuating signal of the capacitive sensor 4 to a supply unit or dental unit or an external controller 16. The unit or the external controller 16 is configured to receive the actuating signal of the capacitive sensor 4 or a control signal triggered by the actuating signal, to process it if necessary, and to control an actuator 13 on the basis of the received actuating signal. For this purpose, the unit or the controller 16 generates an actuator control signal that can be transmitted to the actuator 13 via the coupling device or transmission device 15.

According to the alternative embodiment of FIG. 6, an internal controller 25 is provided in the treatment instrument 1, which is configured to receive the actuating signal of the capacitive sensor 4 or a control signal triggered by the actuating signal, to process it if necessary and to control an actuator 13 on the basis of the received actuating signal. For this purpose, the internal controller 25 generates an actuator control signal that can be transmitted to the actuator 13.

Optionally, the actuating signal of the capacitive sensor 4 or the actuator control signal of the internal controller 25 can be transmitted via the coupling device or transmission device 15 to a supply unit or dental unit or an external controller 16. The unit or external controller 16 is configured to receive the actuating signal or actuator control signal, process it if necessary, and control an external device based on the received signal. The external device comprises, for example, an external display device on which the actuation of the actuator 13 or a value of an operating parameter of the actuator 13 is displayed.

The embodiments described or illustrated serve in particular to illustrate the invention. The features disclosed in one embodiment are therefore not limited to this embodiment but can be combined individually or together with one or more features of one of the other embodiments.

What is claimed is:

1. A medical or dental hand-held treatment instrument, comprising
a single-part or multi-part outer sleeve,
at least one controlling element having at least one capacitive sensor and a sensor surface which is provided on the outer sleeve and is operatively connected to the capacitive sensor, wherein the controlling element generates an actuating signal when the sensor surface is touched or approached, and
an indicating device which is arranged on the hand-held treatment instrument and which at least one of (i) indicates the sensor surface or (ii) indicates the generation of an actuating signal as a result of contact with the sensor surface or approach to the sensor surface.

2. The medical or dental hand-held treatment instrument according to claim 1, wherein
at least a part of the indicating device is provided on the sensor surface and/or adjacent to the sensor surface.

3. The medical or dental hand-held treatment instrument according to claim 1, wherein
at least a part of the indicating device forms the sensor surface.

4. The medical or dental hand-held treatment instrument according to claim 1, wherein
the indicating device is configured to generate vibrations.

5. The medical or dental hand-held treatment instrument according to claim 4, wherein
the indicating device for generating vibrations is operatively connected to the sensor surface for transmitting vibrations to the sensor surface.

6. medical or dental hand-held treatment instrument according to claim 4, wherein
the indicating device for generating vibrations generates vibrations that are acoustically perceptible to a user.

7. The medical or dental hand-held treatment instrument according to claim 1, wherein
the indicating device is configured to deform the sensor surface in a manner perceptible to a user.

8. The medical or dental hand-held treatment instrument according to claim 1, wherein
the indicating device emits light that is visible to a user.

9. The medical or dental hand-held treatment instrument according to claim 8, wherein
the visible light emitted by the indicating device passes through the sensor surface or through a light emitting surface separated or spaced from the sensor surface.

10. The medical or dental hand-held treatment instrument according to claim 1, comprising
a circuit board arranged inside the treatment instrument and on which at least part of the indicating device is arranged.

11. The medical or dental hand-held treatment instrument according to claim 1, comprising
control electronics arranged in the treatment instrument which are connected to the capacitive sensor and the indicating device and are configured to receive the actuating signal of the capacitive sensor and to control the indicating device on the basis of the received actuating signal to indicate a contact with the sensor surface or an approach to the sensor surface.

12. The medical or dental hand-held treatment instrument according to claim 11, wherein
the control electronics are further connected to an actuator and are provided for controlling the actuator on the basis of the received actuating signal of the capacitive sensor.

13. The medical or dental hand-held treatment instrument according to claim 1, wherein
a transmission device which is configured to transmit the actuating signal, or a control signal triggered by the actuating signal to an external controller.

14. The medical or dental hand-held treatment instrument according to claim 1, wherein
the indicating device comprises a marking on the surface of the outer sleeve that is visible to a user.

15. The medical or dental hand-held treatment instrument according to claim 14, wherein
the marking comprises a color marking.

16. The medical or dental hand-held treatment instrument according to claim 1, wherein
the indicating device comprises a marking on the surface of the outer sleeve that is tactile to a user.

17. The medical or dental hand-held treatment instrument according to claim 16, wherein
the marking comprises at least one depression and/or elevation for haptic perception.

18. The medical or dental hand-held treatment instrument according to claim 14, wherein
the marking is fixed on or applied to the outer sleeve and/or the sensor surface.

19. The medical or dental hand-held treatment instrument according to claim 14, wherein
the marking is formed on or by the outer sleeve and/or sensor surface.
